(19) United States
(12) Patent Application Publication
(10) Pub. No.: US 2025/0369834 A1
(43) Pub. Date: Dec. 4, 2025

(54) MECHANICAL SYSTEM HAVING AN OIL SAMPLING DEVICE

(71) Applicant: AIRBUS HELICOPTERS, Marignane Cedex (FR)

(72) Inventor: Eric FABRIGAT, Velaux (FR)

(73) Assignee: AIRBUS HELICOPTERS, Marignane Cedex (FR)

(21) Appl. No.: 19/076,149

(22) Filed: Mar. 11, 2025

(30) Foreign Application Priority Data
May 31, 2024 (FR) FR2405665

(51) Int. Cl. G01N 1/14; G01N 1/10; G01N 33/28

(52) U.S. Cl. CPC G01N 1/14; G01N 33/28; G01N 2001/1037

(57) ABSTRACT

A mechanical system having a casing delimiting an internal volume containing oil. The mechanical system comprises a sampling device for sampling at least part of the oil. The sampling device comprises at least one pipework integral with the casing, the pipework extending from an inner end located in the internal volume to a neck comprising an outer end open to an external environment located outside the mechanical system. The sampling device comprises a closure that can be operated by an operator to close off the neck. The sampling device comprises, in the neck, a pollution barrier providing a restricted passage through which a sampling pipe can pass.

### CROSS REFERENCE TO RELATED APPLICATION

This application claims the benefit of FR 24 05665 filed on May 31, 2024, the disclosure of which is incorporated in its entirety by reference herein.

### TECHNICAL FIELD

The present disclosure relates to a mechanical system having an oil sampling device. For example, such a mechanical system may be an aircraft power transmission box, and in particular may be a power transmission box set in motion by at least one engine in order to rotate at least one rotor of a rotorcraft.

### BACKGROUND

By way of illustration, a mechanical system may comprise movable elements lubricated and/or cooled with oil. The mechanical system then includes a filling port in order to enable the oil to be introduced into an internal volume of the mechanical system. Outside of this filling phase, the filling port can be closed by a cap. In addition, a removable strainer can be disposed in the filling port to limit the risk of introducing pollutants into the internal volume of the mechanical system together with the oil.

Maintenance procedures may include sampling part of the oil for analysis purposes, for example to detect flaking. An operator then removes the cap and optionally the strainer, then introduces a sampling pipe into the mechanical system, via the filling port, in order to sample part of the oil.

Although such a procedure is effective, pollutants may possibly be accidentally introduced into the mechanical system during this maintenance operation. In addition, it may be difficult to determine precisely where in the mechanical system the oil was taken from, which may ultimately complicate the analysis of the results.

In addition, compression glands and towel holder devices are known that do not belong to the technical field of maintenance devices for a mechanical system.

### SUMMARY

An object of the present disclosure is therefore to provide an innovative mechanical system that makes it possible to sample oil for maintenance purposes while limiting the risk of pollution.

Documents CN 218180400 U, CN 217717083 U, JP 2017110667 A, and US20180355987 Al are known.

In particular, document CN 218180400 U describes a single pipe passing through a screw fastener, the pipe comprising a counterweight.

The disclosure thus relates to a mechanical system having a casing delimiting an internal volume containing oil.

This mechanical system comprises a sampling device for sampling at least part of the oil, the sampling device comprising at least one pipework integral with the casing, the pipework extending from an inner end located in the internal volume to a neck comprising an outer end open to an external environment and located outside the mechanical system, the sampling device comprising a closure that can be operated by an operator to close off the neck, the sampling device comprising, in the neck, a pollution barrier providing a restricted passage, or even a single passage, through which a sampling pipe can pass.

The sampling pipe may be part of the sampling device. In particular, the sampling pipe may be part of a sampling kit further comprising a suction pump capable of sucking oil into the sampling pipe and/or a container capable of collecting the oil sucked by the sampling pipe.

The term “pipework” refers to a fluidic connection that may comprise one or more pipes, one or more structural ducts of the casing, and/or one or more hydraulic connectors. For example, the pipework may comprise a duct that is provided in the casing, and a sampling port that is attached to the casing so as to open onto the duct and form the neck. According to another example, the pipework comprises a rigid duct attached to the casing by conventional means of screwing, gluing, welding, riveting or the like.

Therefore, the mechanical system includes a pipework that is closed by the closure at rest, i.e., outside of an oil sampling phase. During such an oil sampling phase, an operator operates the closure to free access to the internal volume. The pollution barrier then limits the risk of introducing pollutants into the internal volume.

Indeed, the pollution barrier restricts a passage surface of the pipework to a restricted passage. The expression “restricted passage” means that the passage has an area less than the cross-sectional area of the pipework housing the pollution barrier. Indeed, at rest, this cross-section has an area equal to the area of the restricted passage and the area covered by the pollution barrier, or even to the area of a member securing the pollution barrier to the pipework. The operator then introduces the sampling pipe into the pipework by passing it through the pollution barrier via the restricted passage. Oil can then be extracted through this sampling pipe for analysis purposes. The operator can then remove the sampling pipe and reposition the closure.

In use therefore, such a sampling device limits the risk of accidental pollution of the oil in the internal volume, due to the pollution barrier arranged in the pipework between the sampling pipe and this pipework. At rest, the closure also protects this internal volume by completely closing off the pipework.

In addition, the pipework also serves as a guide by guiding the movement of the sampling pipe in the internal volume. In order to take an oil sample in a specific area, the inner end of the pipework may be located in the desired area. This area may be located substantially at mid-height of the volume of oil within the casing so that the oil withdrawn is representative of the oil circulating in the mechanical system, being for example free of heavy particles that rest at the bottom of the casing. Alternatively, this area can be chosen to detect all kinds of pollutants, for example water or others, in particular locations of the mechanical system. The mechanical system may further comprise several pipeworks in order to be able to probe several areas and/or sampling pipes having different lengths may be introduced into the same pipework for this purpose.

The pipework may also be more rigid than the sampling pipe, the rigidity of the pipework allowing this pipework not to deform when inserting the sampling pipe. On the contrary, the flexibility of the sampling pipe enables it to be easily inserted and moved in the pipework.

Consequently, the sampling device according to the disclosure proves to be relatively simple and makes it possible to carry out an accurate sampling of oil within the mechanical system, while limiting the risk of introducing pollutants, in particular dust, into the mechanical system during such an operation.

The mechanical system may also comprise one or more of the following features, taken individually or in combination.

According to one possibility, the pollution barrier may be disposed in the pipework between the outer end and the inner end.

The pollution barrier is then not located in the outer end. Such a feature limits, for example, the risk of degrading this pollution barrier during use.

According to one possibility compatible with the preceding possibilities, said pollution barrier may be removable.

The pollution barrier may, by way of illustration, be disposed on a ring inserted into the pipework. For example, this ring may be screwed to the pipework, may comprise tabs sliding in grooves of the pipework, may comprise a stop resting on the outer end, or the like.

Irrespective of the variant, the pollution barrier can be connected to the pipework via a reversible system. This pollution barrier can then be easily replaced in the presence of deterioration.

Alternatively, the pollution barrier may be attached by conventional means to the pipework.

Optionally, a grid may be disposed in the pipework downstream of the pollution barrier, in a direction towards the internal environment, to prevent the introduction of part of this pollution barrier into the pipework in the event of deterioration.

According to one possibility compatible with the preceding possibilities, the pollution barrier may comprise an elastic membrane delimiting said restricted passage.

The elastic membrane may comprise an orifice forming the above-mentioned restricted passage. For example, the elastic membrane is perforated to form the passage, optionally at its center. With respect to an axis passing inside the neck, the elastic membrane may extend radially from the internal wall of the pipework to the passage. The membrane may thus comprise an annular outer section substantially adjoining the inner wall, and a perforated inner section to form the passage. The passage may advantageously have a circular shape, or alternatively a cross shape, a star shape, etc.

Such an elastic membrane may delimit a passage smaller than the sampling pipe.

As a result, the risk of introducing pollutants is reduced.

In addition, the elastic membrane may be used to hold the sampling pipe in position during oil sampling.

According to one possibility wherein the sampling device comprises the sampling pipe, the elastic membrane can stretch when the sampling pipe is introduced, widening said passage, the elastic membrane matching the sampling pipe.

When the sampling pipe is inserted, this sampling pipe exerts a force on the elastic membrane. The elastic membrane deforms without tearing, which enables the dimensions of the passage to increase until the sampling pipe passes through the passage, this passage being able to be adapted to the sampling pipe.

Therefore, the passage may be of very small dimensions at rest, and may be largely, or even completely, filled by the sampling pipe in use. The risk of introducing pollutants is reduced.

Optionally, the elastic membrane may comprise a material from the group of elastomers, or even in particular from the group of rubbers.

Such a material makes it possible to obtain a membrane that is sufficiently rigid at rest to provide an obstacle to pollutants, and sufficiently flexible to be able to be deformed and passed through by the sampling pipe in use.

According to one possibility compatible with the preceding possibilities, since the sampling device may comprise the sampling pipe described above, the sampling pipe may be thinner, i.e., narrower, than the pipework in order to be able to be introduced into the pipework and pass through said pollution barrier via the passage.

This feature enables the sampling pipe to be inserted into the pipework.

In addition, the pipework may have an inner diameter larger than the outer diameter of the sampling pipe, but smaller than the diameter of a conventional filling port.

According to one possibility compatible with the preceding possibilities, the sampling device being able to comprise the sampling pipe described above, the sampling pipe may have a length calibrated with respect to a length of the pipework in order to exit on either side of the pipework and in particular outside the pipework on the side of the external environment by a predetermined protruding length during an oil sampling phase.

The sampling device then makes it possible to position the sampling pipe in the internal volume in a reproducible manner. The sampling pipe protrudes from the pipework by a determined length, substantially identical at each sampling, in order to reach the targeted sampling area. By way of illustration, the pipework may have a length of 30 centimeters, and the sampling pipe may have a length of 50 centimeters. By indicating that the sampling pipe should protrude 15 centimeters into the outside environment, the sampling device ensures that the sampling pipe will protrude substantially from the pipework into the internal volume by a length of 5 centimeters. The end of the sampling pipe present in the internal volume will thus be substantially always in the same place.

According to one possibility compatible with the preceding possibilities, the sampling device may comprise the sampling pipe described above, the sampling pipe may comprise at least one marking or protrusion configured to place the sampling pipe at a predetermined position relative to the pipework, in particular relative to the open outer end of the pipework.

This marking or protrusion may be positioned substantially at the outer end of the pipework when inserting the sampling pipe. This marking or protrusion makes it possible to place the sampling tube precisely and reproducibly.

According to one possibility compatible with the preceding possibilities, the closure may comprise a lid that is hinged to the neck or screwed to the neck to close off this neck.

Such a lid can effectively close off the outer end.

The lid and/or the neck may comprise a sealing means, such as a gasket for example, so that the lid can close off the pipework in an oil-tight manner for example.

Additionally of alternatively, the closure may include a cap screwed to an outer wall of the neck.

A conventional cap can close off the neck.

The cap and/or the neck may comprise a sealing member, such as a gasket, for example, so that the cap can close off the pipework in an oil-tight manner, for example.

Optionally, the closure comprises the above-mentioned cap and lid. The cap can cover the lid when this cap is screwed to the external wall of the neck.

Thus, the cap is removed during the initiation of the oil sampling phase, and the lid is only moved before the sampling pipe is introduced to minimize the risk of introducing pollutants into the mechanical system.

According to one possibility compatible with the preceding possibilities, the mechanical system may comprise a filling port separate from the sampling device for introducing the oil into the internal volume.

Optionally, a horizontal plane passes through the outer end, this horizontal plane passing through the filling port or being above the filling port, while the mechanical system has a pitch angle and a roll angle within a predetermined pitch range and a predetermined roll range, respectively.

The closure of the sampling device is at least at the same level, in altitude, as the filling port. This characteristic makes it possible to have the same level of risk of overflow via the pipework as via the filling port. Optionally, the closure is placed so as to facilitate the oil sampling phase. For example, the closure may be placed next to the filling port.

In another aspect, the mechanical system may be a power transmission box.

In another aspect, a vehicle may have a mechanical system according to the disclosure. For example, a rotorcraft may comprise at least one engine moving a mechanical system according to the disclosure, the mechanical system moving at least one rotor.

### BRIEF DESCRIPTION OF THE DRAWINGS

The disclosure and its advantages appear in greater detail from the following description of examples given by way of illustration with reference to the accompanying figures, wherein:

FIG. 1 is a diagram illustrating a mechanical system according to the disclosure;

FIG. 2 is a diagram illustrating a mechanical system having a closed-off oil sampling device;

FIG. 3 is a diagram illustrating the mechanical system of FIG. 2 before insertion of a sampling pipe;

FIG. 4 is a diagram illustrating a mechanical system of FIG. 2 during insertion of the sampling pipe; and

FIG. 5 is a diagram illustrating a mechanical system of FIG. 2 during extraction of the sampling pipe.

### DETAILED DESCRIPTION

Elements present in more than one of the figures are given the same references in each of them.

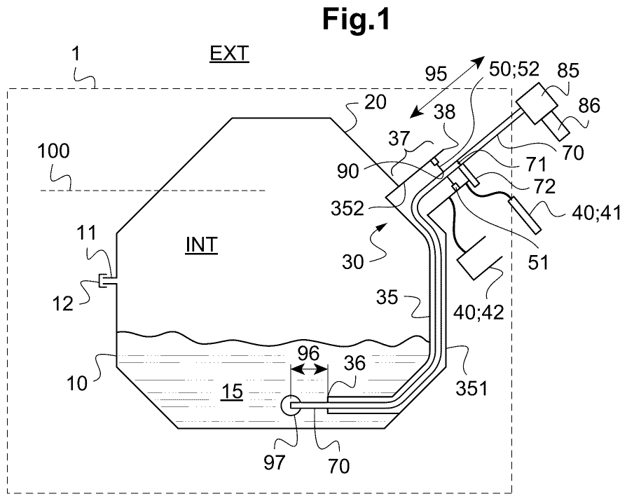

FIG. 1 illustrates a mechanical system 10 according to the disclosure. The mechanical system 10 has a casing 20 delimiting an internal volume INT containing oil 15. The casing 20 may comprise one or more cases.

This mechanical system 10 may be arranged in a vehicle 1. This vehicle 1 may be an aircraft, or even a rotorcraft, for example.

For example, the mechanical system 10 may comprise at least one movable element (not shown), such as a shaft, a pinion, a wheel, bearings or the like, lubricated and/or cooled by a system for powerful lubrication and/or cooling of oil 15. For example, the mechanical system 10 may be a power transmission box.

Irrespective of its arrangement, the mechanical system 10 may comprise a filling device having a filling port 11 to allow oil 15 to be introduced into the internal volume INT. The filling device may comprise a filling cap 12 for closing off the filling port 11 and/or a strainer, for example.

Irrespective of these aspects, the mechanical system 10 comprises a sampling device 30 that has the function of enabling at least part of the oil 15 to be sampled, for analysis purposes for example. This sampling device 30 is distinct from the above-described optional filling device.

The sampling device 30 comprises a pipework 35 fixed relative to the casing 20. The pipework 35 may be attached to the casing 20 or may be at least partially provided using casing elements 20.

This pipework 35 extends from an inner end 36 situated in the internal volume INT to an outer end 38 opening onto an external environment EXT located outside the mechanical system 10. The outer end 38 may be located in the external environment EXT.

In particular, the outer end 38 is present in an end area of the pipework 35 called the “neck 37”. For example, this neck 37 may be at least partially located in the external environment EXT.

According to the example illustrated, the pipework 35 comprises a structural duct 351 integrated into the casing 20 opening into a sampling port 352 attached to the casing 20.

Optionally, the neck 37 may be located at the same altitude or at an altitude higher than the altitude of the filling port 11. A horizontal plane 100 passing through the outer end 38 may then either pass through the filling port 11 or pass above the filling port 11, while the mechanical system 10 has a pitch angle and a roll angle within a predetermined pitch range and a predetermined roll range, respectively.

In addition, the sampling device 30 comprises a closure 40 that can be operated by an operator in order to isolate the inside of the pipework 35 from the external environment EXT.

The closure 40 may comprise a lid 41. The lid 41 can be moved from an open position, allowing access to the interior of the pipework 35, to a closed position wherein the lid 41 closes the outer end 38. Optionally, the lid may include a sealing means 80, such as a gasket pressed against the neck when the lid closes the pipework. Alternatively, the neck may comprise such a sealing means 80 pressed against the lid when the lid closes the pipework.

According to the example of FIG. 1, the lid 41 can be screwed to the pipework 35 in the closed position. Optionally, the lid 41 may be connected by a link to the pipework 35 or to the casing 20 so as not to be lost in an open position.

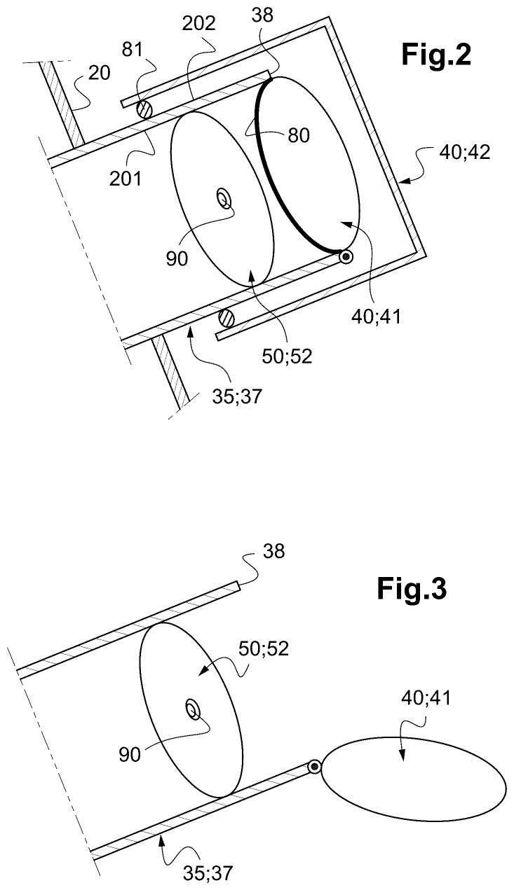

According to the example of FIGS. 2 to 5 described below, the cap 41 may be hinged to the neck 37 so as to be able to move in rotation relative to the pipework 35.

Alternatively or additionally and with reference to FIG. 1, the closure 40 may comprise a cap 42. Such a cap 42 can be screwed to an outer wall 202 of the neck 37. Optionally, a link may connect the cap 42 to the pipework 35 or to the casing 20 so as not to lose the cap 42 when it is removed. Optionally, the cap may comprise a sealing member 81, such as a gasket pressed against the neck when the cap closes the pipework. Alternatively, the neck may comprise such a sealing member 81 pressed against the cap when the cap closes the pipework.

In another aspect, the sampling device 30 may include a removable sampling pipe 70. This sampling pipe 70 can be inserted into the pipework 35 during an oil sampling phase.

The sampling pipe 70 may be part of a kit further comprising a pump 85 and/or a container 86. The pump 85 may be attached to the sampling pipe 70 and may open onto the container 86.

Furthermore, the sampling pipe 70 is thinner than the pipework 35 in order to be able to be introduced into this pipework 35. For example, the sampling pipe 70 has an external diameter less than an internal diameter of the pipework 35.

Furthermore, the sampling pipe 70 may be flexible, whereas the pipework 35 may be rigid.

In another aspect, the sampling pipe 70 may have a length calibrated with respect to a length of the pipework 35. This calibrated length may be defined so that the sampling pipe 70 exits on either side of the pipework 35 over predetermined distances 95, 96. Thus, the sampling pipe 70 has a length 96 in the internal volume INT at the outlet of the inner end 36, and a protruding length 95 outside the pipework 35 on the external medium side EXT. During a sampling phase, an operator introduces the sampling pipe 70 into the pipework 35, taking care to have the desired protruding length 95. This feature ensures that the sampling pipe 70 reaches the desired sampling area 97 in the internal volume INT.

Additionally or alternatively, the sampling pipe 70 has a marking 71, such as a colored line, for example. The operator can move the sampling pipe 70 relative to the pipework 35 so as to place the marking 71 in a predetermined position relative to the pipework 35, for example at the outer end 38 according to the example illustrated.

Additionally or alternatively, the sampling pipe 70 has a protrusion 72 forming a stop. Such a protrusion 72 can both position the sampling pipe 70 correctly, and optionally prevent this sampling pipe 70 from falling into the pipework 35, and consequently into the mechanical system 10.

According to another aspect, the sampling device 30 comprises a pollution barrier 50 arranged in the neck 37 to limit the risk of polluting the internal volume INT, in particular during an oil sampling phase. The pollution barrier 50 restricts an internal passage surface of the pipework 35 to a passage 90 through which the sampling pipe 70 can pass.

This pollution barrier 50 may be disposed in the pipework 35 between the outer end 38 and the inner end 36.

The pollution barrier 50 may comprise an elastic membrane 52 that is disposed in the pipework 35, and in particular in the neck 37.

This elastic membrane 52 may be removable. For example, the elastic membrane 52 is integral with a ring 51 of the pollution barrier 50. This ring 51 can be screwed to an internal wall of the neck 37. According to another example, this ring 51 may comprise radial lugs sliding in slides of the pipework 35.

According to another example, the elastic membrane 52 is secured to the pipework 35, for example by gluing.

Irrespective of its embodiment, the pollution barrier 50 extends from the pipework 35 and delimits the restricted passage 90.

In other words, the pollution barrier 50 is integral with the neck 37 so that the elastic membrane 52 reduces a passage surface within the pipework 35 to the single passage 90. For example, the elastic membrane 52 has an annular shape, or a disk shape having a cut-out forming said passage 90. Optionally, the passage 90 provided may have smaller dimensions than the sampling pipe 70.

Optionally, the elastic membrane 52 is obtained with a material from the group of elastomers.

FIGS. 2 to 5 illustrate the operation of the disclosure with a closure having a cap and a lid. A similar operation is in fact obtained in the presence of only the cap or the lid, the operations related to the cap or the lid being identical to the operations that follow.

At rest and as illustrated in FIG. 2, the closure 40 prevents pollutants from reaching the elastic membrane 52. According to the illustrated example, the closure 40 comprises a lid 41 and a cap 42. The lid 41 is in a closed position and covers the outer end 38. In addition, the cap 42 is screwed to the neck 37.

In order to take an oil sample and as illustrated in FIG. 3, an operator operates the closure 40. According to the example given, the operator unscrews the cap 42. The operator then operates the lid 41 to position it in an open position just before introducing the sampling pipe 70. Once the lid 41 is moved into its open position, the elastic membrane 52 limits the risk of pollutants entering the pipework 35 and the mechanical system 10, since the passage surface in the neck 37 is reduced to the area of the passage 90.

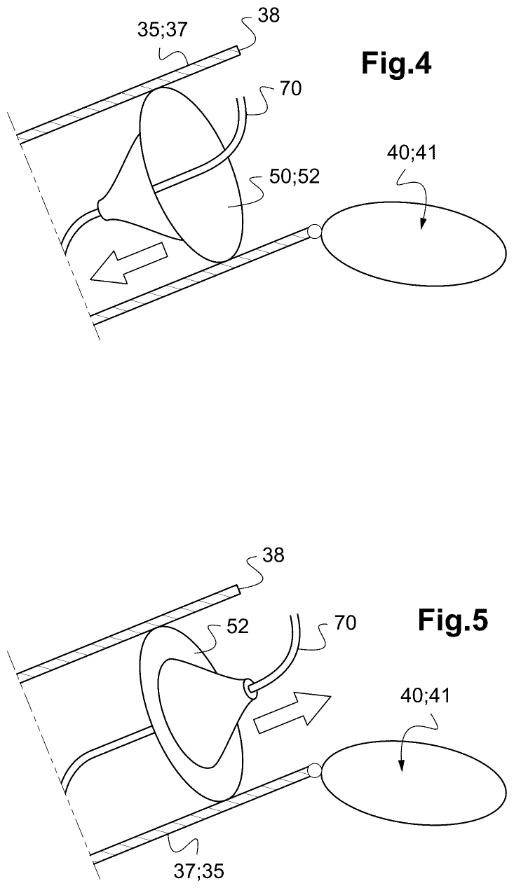

As illustrated in FIG. 4, the operator then inserts the sampling pipe 70. The sampling pipe 70 deforms the elastic membrane 52 so as to enlarge the passage 90. The sampling pipe 70 then passes through the elastic membrane 52. Moreover, the elastic membrane 70 matches the shape of the sampling pipe 70 leaving little or no space between the elastic membrane 52 and the sampling pipe 70. The elastic membrane 52 can also prevent the sampling pipe 70 from falling into the pipework 35 and, as a result, into the mechanical system 10.

The operator can then appropriately position the sampling pipe 70 relative to the pipework, for example by means of the above-mentioned marking and/or protrusion. The operator can, if necessary, actuate the pump 85 connected to the sampling pipe 70 to sample the required oil 15.

At the end of the sampling and as illustrated in FIG. 5, the operator can then pull on the sampling pipe 70 to extract it from the pipework 35. The operator repositions the lid 41 in its closed position and screws the cap 42 to the pipework.

Naturally, the present disclosure may be subjected to numerous variations as to its implementation. Although several embodiments are described above, it should readily be understood that it is not conceivable to identify exhaustively all the possible embodiments. It is of course possible to replace any of the means described with equivalent means without going beyond the ambit of the present disclosure.

What is claimed is:

1. A mechanical system having a casing delimiting an internal volume containing oil,
wherein the mechanical system comprises a sampling device for sampling at least part of the oil, the sampling device comprising at least one pipework integral with the casing, the pipework extending from an inner end located in the internal volume to a neck comprising an outer end open to an external environment located outside the mechanical system, the sampling device including a closure that can be operated by an operator to close off the neck, the sampling device comprising, in the neck, a pollution barrier providing a restricted passage through which a sampling pipe can pass.

2. The mechanical system according to claim 1,
wherein the pollution barrier is disposed in the pipework between the outer end and the inner end.

3. The mechanical system according to claim 1,
wherein the pollution barrier is removable.

4. The mechanical system according to claim 1,
wherein the pollution barrier comprises an elastic membrane delimiting the passage.

5. The mechanical system according to claim 4,
wherein the elastic membrane comprises a material from the elastomer group.

6. The mechanical system according to claim 4,
wherein the sampling device comprises the sampling pipe, the elastic membrane stretching when the sampling pipe is introduced, widening the passage, the elastic membrane matching the sampling pipe.

7. The mechanical system according to claim 1,
wherein the sampling device comprises the sampling pipe, the sampling pipe being thinner than the pipework so as to be able to be introduced into the pipework and pass through the pollution barrier via the passage.

8. The mechanical system according to claim 1,
wherein the sampling device comprises the sampling pipe, the sampling pipe has a length calibrated with respect to a length of the pipework in order to exit on either side of the pipework and in particular outside the pipework on the side of the external environment by a predetermined protruding length during an oil sampling phase.

9. The mechanical system according to claim 1,
wherein the sampling device comprises the sampling pipe, the sampling pipe has at least one marking or protrusion configured to place the sampling pipe at a predetermined position with respect to the pipework.

10. The mechanical system according to claim 1,
wherein the closure comprises a lid hinged to the neck or screwed to the neck in order to close off this neck.

11. The mechanical system according to claim 1,
wherein the closure comprises a cap screwed to an outer wall of the neck.

12. The mechanical system according to claim 10, wherein the closure comprises a cap screwed to an outer wall of the neck and wherein the cap covers the lid when this cap is screwed to the outer wall of the neck.

13. The mechanical system according to claim 1,
wherein the mechanical system comprises a filling port separate from the sampling device for introducing the oil into the internal volume, a horizontal plane passing through the outer end, this horizontal plane passing through the filling port (11) or being above the filling port while the mechanical system has a pitch angle and a roll angle within a predetermined pitch range and a predetermined roll range, respectively.

14. A vehicle having the mechanical system,
wherein the mechanical system is according to claim 1.
